(19) United States
(12) Patent Application Publication
(10) Pub. No.: US 2006/0149371 A1
(43) Pub. Date: Jul. 6, 2006

(54) Intervertebral prosthetic device and method with locking mechanism

(75) Inventors: Gregory C. Marik, Germantown, TN (US); Craig M. Squires, Cordova, TN (US); Eddie F. Ray III, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/010,171

(22) Filed: Dec. 10, 2004

(51) Int. Cl. A61F 2/44

(52) U.S. Cl. 623/17.11

(57) ABSTRACT

A prosthetic device and method, according to which first end plate engages a first vertebral body, and a second end plate engages a second vertebral body. An insert lockingly engages one of the end plates and engages the other end plate in a manner to permit relative movement therebetween.

### BACKGROUND

This invention relates to an intervertebral endoprosthetic device and a method of assembling same and, more particularly, to such a device and method in which a locking mechanism is provided to lock two components of the device.

Spinal discs in the human body may rupture or degenerate to such a degree that the disc has to be surgically removed. In these cases, intervertebral prosthetic devices are available that are designed to be implanted between the adjacent vertebrae to prevent the collapse of the intervertebral space between adjacent vertebrae while maintaining a certain degree of stability and range of pivotal and rotational motion therebetween. Such devices typically include two or more articular elements that are attached to the respective adjacent vertebrae and are adapted to move relative to each other.

Many of these devices, especially those that are inserted in the cervical spine, are either made out of a single component, or are made from multiple components which are permanently fixed together. Therefore, these devices do not offer modularity or revisability without removing the entire assembly.

Although modular systems do exist for use in the lumbar spine, these systems use hinges on the components, or modules, to connect the components. However this type of connection is not robust and/or tends to malfunction, causing disassociation that often results in significant complications.

All patents listed in Table 1 are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the teachings of the present invention.

TABLE 1

| Patent/Publication No. | Patented/Published Date | Inventor |
|---|---|---|
| 5,425,773 | Jun. 20, 1995 | Boyd et al. |
| 5,562,738 | Oct. 8, 1996 | Boyd et al. |
| 6,146,421 | Nov. 14, 2000 | Gordon et al. |
| 2002/0099444 | Jul. 25, 2002 | Boyd et al. |
| 2003/0040799 | Feb. 27, 2003 | Boyd et al. |
| 2004/0002758 | Jan. 1, 2004 | Landry et al. |
| 6,682,561 | Jan. 27, 2004 | Songer et al. |
| 2004/0068318 | Apr. 8, 2004 | Coates et al. |
| 6,726,720 | Apr. 27, 2004 | Ross et al. |
| 2004/0083000 | Apr. 29, 2004 | Keller et al. |

### SUMMARY

According to an embodiment of the invention, an intervertebral prosthesis is provided that includes an insert that is encapsulated in a base member of the prosthesis in a manner to provide minimal opportunity for disassociation.

Various embodiments of the invention discussed below may possess one or more of the above features and advantages, or provide one or more solutions to the above problems existing in the prior art.

### BRIEF DESCRIPTION OF THE DRAWINGS

FIG. 1 is a sectional view of a vertebral column of a human body having a damaged disc.

FIG. 2 is an enlarged partial view of the vertebral column of FIG. 1 and depicting an implanted intervertebral prosthesis according an embodiment of the invention.

FIG. 3 is an exploded isometric view of the prosthesis of FIG. 2

FIG. 4 a partial sectional/partial elevational view showing a step in assembling the prosthesis of FIGS. 2 and 3.

FIG. 5 is an enlarged sectional view of the assembled prosthesis of FIGS. 2 and 3.

FIGS. 6 and 7 are partial exploded isometric view of two alternate embodiments.

FIGS. 8 and 9 are isometric views of two additional alternate embodiments.

### DETAILED DESCRIPTION OF THE PREFERRED EMBODIMENTS

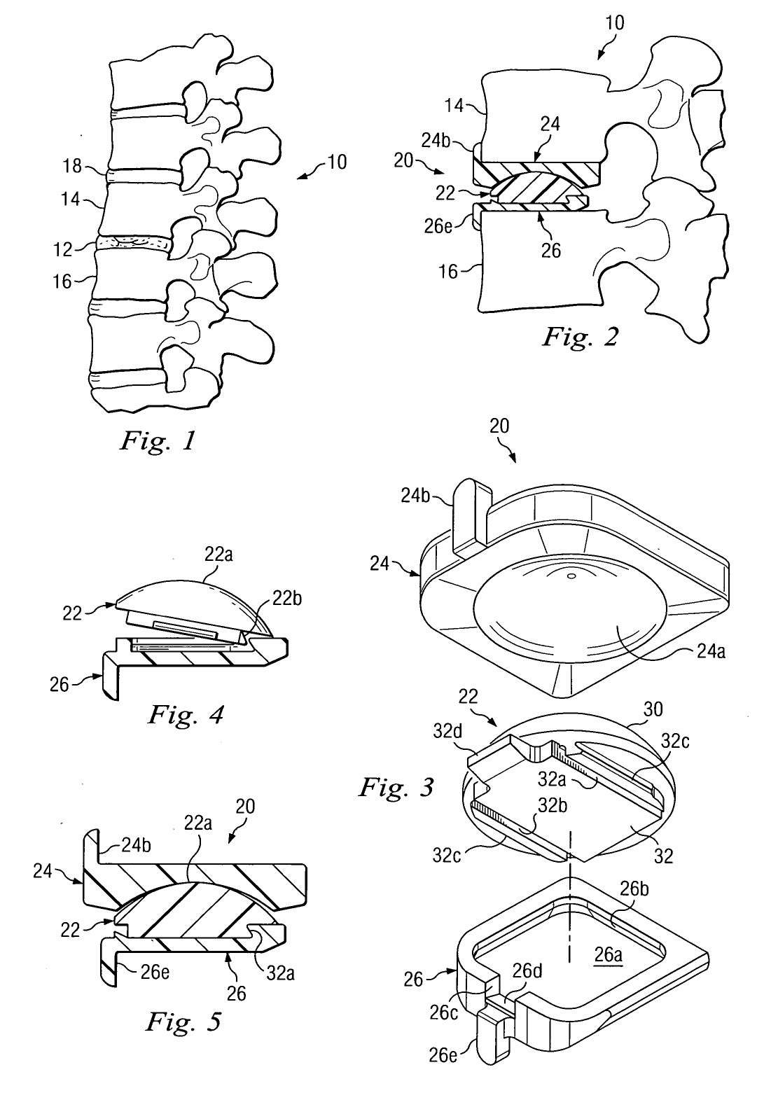

Referring first to FIG. 1, the numeral 10 refers to a vertebral column with a damaged intervertebral disc 12 extending between two adjacent vertebrae 14 and 16 in the cervical, thoracic, or lumbar spine, or in other regions of the vertebral column.

Referring to FIG. 2, it will be assumed that a typical surgical discectomy was performed to remove the disc 12 and create a void between the two intact vertebrae 14 and 16, and that an intervertebral disc prosthesis 20 according to an embodiment of the invention is implanted in the above void.

The prosthesis 20 is shown in detail in FIGS. 3 and 4 and includes an insert 22 interposed between an upper end plate 24 and a lower end plate 26, as viewed in the drawings. The upper end plate 24 is generally rectangular in shape with a curved anterior, or front surface, as viewed in FIGS. 3 and 4, and an upper surface that is either flat or contoured to match the corresponding surface of the vertebrae 14 (FIG. 2). A semi-spherical recess 24a is formed in the lower surface of the end plate 24, and a tab 24b extends upwardly from its anterior end.

The lower end plate 26 is also generally rectangular in shape with a curved anterior end surface, and its upper surface includes a generally rectangular recessed area 26a bounded on its two sides and its end by a groove 26b. An access notch 26c is formed in the anterior end portion of the end plate 26 that extends from the outer surface of the end plate to the recessed area 26a, and a ramp 26d is formed at the base of the notch. A tab 26e extends downwardly from the anterior end of the end plate 26. The lower surface of the end plate 26 is flat as shown, but could be contoured to match the corresponding surface of the vertebrae 16 (FIG. 2).

It is understood that the end plates 24 and 26 are fastened to the vertebrae 14 and 16, respectively, in any conventional manner which would include locating the tabs 24b and 26e over the corresponding outer anterior surface of the vertebrae 14 and 16, respectively, as shown in FIG. 2, to prevent posterior movement of the prosthesis. For example, fasteners, such as screws, or the like, could be driven through any part of the end plates 24 and 26, including the tabs 24b and 26e, and into the vertebrae 14 and 16 to fasten the prosthesis 10 to the vertebrae. According to another example, bone-engaging surfaces, or fins, can be provided on the upper surface of the end plate 24 and the lower surface of the end plate 26, to engage the vertebrae 14 and 16, respectively, to enhance secure implantation of the prosthesis 10.

The insert 22 has a domed upper portion 30 formed integrally with, and extending upwardly from, a substantially rectangular lower portion 32. The domed upper portion 30 fits within the recess 24a of the end plate 24 with minimal clearance to form an articulating joint, permitting rotational movement between the insert 22 and the end plate 24.

Two parallel, longitudinally extending, notches 32a and 32b are provided in the rectangular portion 32 and are spaced slightly inwardly from the respective side walls of the rectangular portion. Thus, each portion of the rectangular portion 32 extending between a notch and its corresponding side wall is relatively flexible. A peripheral dovetail projection 32c extends from the side walls and the posterior wall of the rectangular portion 32, and is sized to extend in the corresponding portions of the groove 26b of the end plate 26.

A tab 32d extends outwardly from the anterior end of the insert 22, and has a flat upper surface that is designed to be engaged by a suitable tool. This enables the surgeon to apply a force to the tab 32d in a direction towards the end plate 26 to aid in locking the insert 22 to the lower end plate 26 in a manner to be described.

The insert 22 is attachable to the lower end plate 26 by initially positioning it over the plate at a slight angle to the plane of the latter end plate as shown in FIG. 4. That portion of the projection 32c that extends from the posterior wall of the rectangular portion 32 of the insert 22 is inserted in the corresponding portion of the groove 26b of the end plate 26. Then the anterior portion of the insert 22 is forced downwardly towards the end plate 26 by engaging a tool with the upper surface of the tab 32d and pressing downwardly. The above-mentioned, relatively flexible, side portions of the rectangular portion 32, including the portions of the projection 32c extending from the side walls of the rectangular portion, snap into the corresponding portions of the groove 26b.

As a result, the insert 22 is locked to the end plate 26 and is substantially encapsulated between the end plates 24 and 26 to considerably reduce the chances for disassociation. Thus, the prosthesis 10 is completed implanted between the vertebrae 14 and 16 as shown in FIG. 5, and the assembly formed by the connected insert 22 and end plate 26 can rotate relative to the end plate 24.

The insert 22 is modular, and as such, can be revised, or replaced, by another insert of a different size, design, or the like. To this end, the insert 22 can be unlocked from the lower end plate 26 by inserting a proper tool (not shown) through the notch 26c and, using the ramp 26d as a guide, wedging the tool between the lower surface of the rectangular portion 32 of the insert 22 and the upper surface of the end plate 26. The tool can then be pivoted about the ramp 26d to force the insert in a direction away from the end plate 26 to effect the separation. The insert 22 can then be completely disconnected from the end plate 26 by sliding the insert anteriorly relative to the end place 26. This permits the insert 22 to be replaced while maintaining the end plates 24 and 26 in their original positions, secured to the vertebrae 14 and 16.

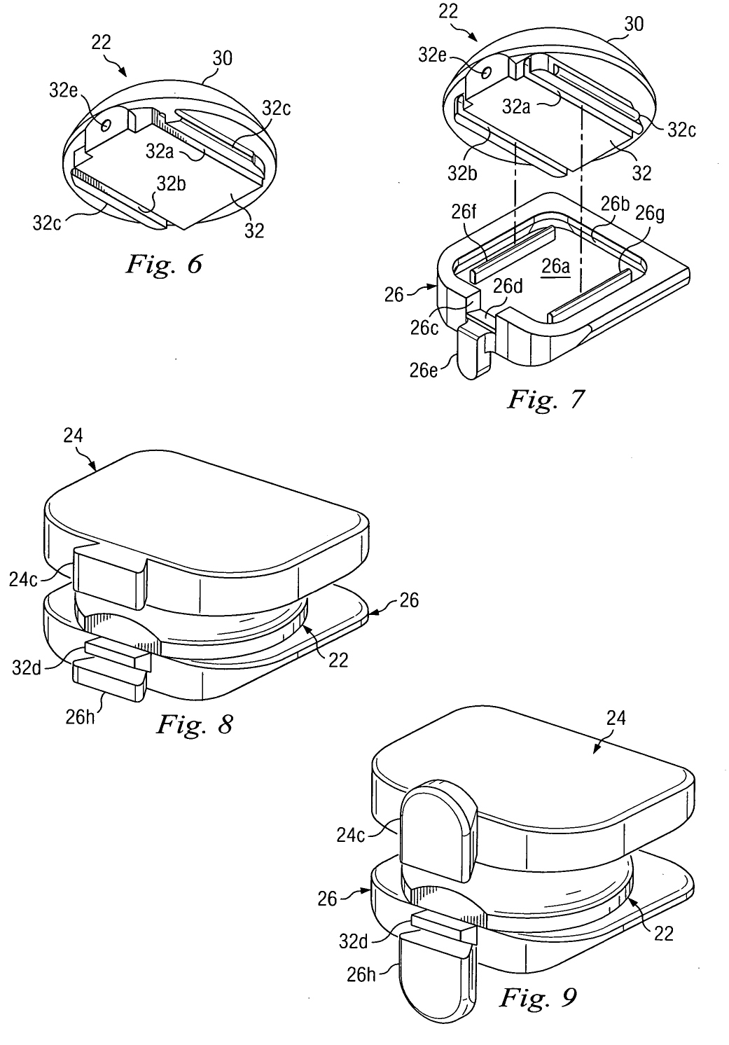

FIG. 6 depicts an alternate embodiment of the invention and includes structure and components of the previous embodiment that are given the same reference numerals. According to the embodiment of FIG. 6, the tab 32d is eliminated and an opening 32e is formed in a flat end portion of the rectangular portion 32 for receiving a tool, for forcing the insert 22 towards the end plate 26 in the assembly step discussed above. Otherwise, the embodiment of FIG. 6 is identical to that of FIGS. 2-5.

The embodiment of FIG. 7 is similar to that of FIGS. 2-5 and identical structure and components are given the same reference numerals. According to the embodiment of FIG. 7, two spaced parallel rails 26f and 26g project upwardly from the recessed area 26a of the lower end plate 26, and are adapted to extend in the notches 32a and 32b respectively. In this context, the depths of the notches 32a and 32b can be increased, when compared to their depths in the embodiment of FIGS. 2-5. Thus, when the insert 22 is locked to the end plate 26 as described above in connection with the embodiment of FIGS. 3-5, the rails 26f and 26g extend in the notches 32a and 32b, respectively, to further secure the insert to the end plate. Otherwise, the embodiment of FIG. 7 is identical to that of FIGS. 3-5 with the understanding that the opening 32b of the embodiment of FIG. 7 could be replaced by the tab 32d of the embodiment of FIG. 3.

The embodiments of FIGS. 8 and 9 are similar to the embodiment of FIGS. 2-5 an identical structure and components are given the same reference numerals. According to the embodiment of FIG. 8, the tabs 24b and 26e on the end plates 24 and 26, respectively, of the embodiment of FIGS. 2-5 are eliminated, and two lugs 24c and 26h are provided on the anterior ends of the respective plates. The lugs 24c and 26h are dove-tailed to form a trapezoidal cross section to enable a corresponding tool to grip them to assist the surgeon in manipulating the prosthesis 10 relative to the vertebrae 14 and 16. Although the tab 32d is shown on the insert 22 in the embodiment of FIG. 8, it is understood that the opening 32d of FIG. 6 could be provided. Also, the rails 26f and 26g of the embodiment of FIG. 7 could be provided, or left off, of the embodiment of FIG. 8. Otherwise the embodiment of FIG. 8 is identical to those of the previous embodiments.

The embodiment of FIG. 9 is identical to that of FIG. 8 with the exception that the lug 24c is extended upwardly as viewed in the drawing, and the lug 26h is extended downwardly. Thus, in addition to providing gripping surfaces as discussed above in connection with the embodiment of FIG. 7, the lugs 24c and 26h also perform the function of the tabs 24b and 26e of the embodiment of FIGS. 2-5. It is understood that, although the tab 32d is shown on the insert 22 in the embodiment of FIG. 9, the opening 32e of FIG. 6 could be provided. Also, the rails 26f and 26g of the embodiment of FIG. 7 could be provided, or left off, of the embodiment of FIG. 9. Otherwise the embodiment of FIG. 9 is identical to those of the previous embodiments.

In all of the above embodiments, the end plates 24 and 26 and may be formed of any suitable biocompatible material including, titanium alloys, cobalt-chrome alloys, stainless steel, alumina, zirconia, polycrystalline diamond, pyrolytic carbon, and the insert 22 can be formed of ultra-high molecular weight polyethylene (UHMWPE), polyetheretherketone (PEEK cross-linked UHMWPE), or other suitable materials. The surfaces of the end plates 24 and 26 and/or the insert 22 may include features or coatings which enhance the purchase or ingrowth qualities of the prosthesis 10. For example, a plazma or biocompatible and osteoconductive material, such as hydroxyapatite (HA), may coat all or a portion of one or more of the above surfaces. Other suitable coatings or treatments may include a porous bead coating, a porous mesh coating, osteogenic peptide coating, growth factor coating, rh-BMP coating, and/or grit blasting. The respective surfaces of the domed portion 30 of the insert 22 and the recessed portion 24a of the upper end plate 24 may be selected or treated to be sufficiently durable to provide acceptable wear characteristics as a result of their articulating movement.

Variations

It is understood that variations may be made in the foregoing without departing from the invention and examples of some variations are as follows:

(1) The prosthesis 10 can be implanted between body portions other than vertebrae.

(2) The prosthesis 10 can be inserted between two vertebrae following a corpectomy in which at least one vertebrae has been removed.

(3) The insert 22 may vary somewhat in shape, size, composition, and physical properties, depending upon the particular area in which the prosthesis is implanted, and more particularly, the space between the end plates 24 and 26 after they have been connected to the vertebrae 14, and 16, respectively.

(4) The upper domed portion of the insert 22 can be replaced by a protrusion of a different shape.

(5) The dome could be formed on the lower surface of the end plate 24 and the corresponding recess formed in the upper surface of the insert 22.

(6) The prosthesis 10 can be implanted between the vertebrae 14 and 16 in a position that is inverted from the position shown in FIG. 2. In this case the end plate 26 would be connected to upper vertebrae 14 and the end plate 24 connected to the vertebrae 16, in which case one or both of the end plates 24 and 26 could vary in shape.

(7) In the embodiment of FIG. 7, rather than provide the rails 26f and 26g in the end plate 26 and the corresponding notches 32c and 32d in the insert 22, the rails could be provided in the insert and the notches in the end plate.

(8) A lug (24c or 26h) can be provided on one of the end plates 24 and 26 and not on the other.

(9) The opening 32d in the embodiment of FIGS. 2-5 may be eliminated.

(10) The spatial references made above, such as “under”, “over”, “between”, “upper”, “lower”, etc. are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. An intervertebral implant comprising:
a first end plate for engaging a first vertebrae;
a second end plate for engaging a second vertebrae and having a groove; and
a insert engaging the first end plate in a manner to permit relative movement therebetween, and having a projection adapted to extend in the groove to lockingly engage the insert with the second end plate and to be manually removed from the groove to permit the insert to be disengaged from the second end plate.

2. The implant of claim 1 wherein the second end plate has a recessed portion for receiving the insert and bounded on at least one side by the groove.

3. The implant of claim 2 wherein the rear portion of the recessed portion is bounded by the groove and wherein the projection is formed on the rear portion of the insert.

4. The implant of claim 2 wherein the rear portion and the sides of the recessed portion are bounded by the groove.

5. The implant of claim 4 wherein the projection also extends from the sides of the insert.

6. The implant of claim 4 wherein the insert is lockingly engaged to the second end plate by positioning the insert at an angle to the second end plate with the projection on the rear end of the insert extending in the groove in the rear portion of the second end plate, and then pressing the front portion of the insert portion towards the second end plate so that the projections on the sides of the insert extend in the groove in the sides of the second end plate.

7. The implant of claim 1 wherein the relative movement is a rotation.

8. The implant of claim 1 further comprising a tab extending from the front end of the first end plate and adapted to engage the corresponding end of the first vertebrae, and a tab extending from the front end of the second end plate and adapted to engage the corresponding end of the second vertebrae.

9. The implant of claim 1 wherein one of the first end plate and the insert has a semi-spherical recess formed therein and wherein the other of the first end plate and the insert has a dome that extends in the recess to permit the relative movement.

10. The implant of claim 1 wherein the first end plate has a semi-spherical recess formed therein and the insert has a dome that extends in the recess to permit the relative movement.

11. The implant of claim 10 wherein the insert also has a rectangular portion extending from the dome, the projecting portion projecting from the rectangular portion.

12. The implant of claim 1 wherein a notch is formed in one of the insert and the second end plate and at least one rail is formed on the other of the insert and the second end plate for extending in the notch.

13. The implant of claim 12 wherein there are two spaced notches and two rails for respectively extending in the grooves.

14. The implant of claim 1 wherein the second end plate has a recessed portion and further comprising a notch formed in the front end of the second end plate for permitting a tool to extend in the recessed portion.

15. The implant of claim 14 further comprising a ramp formed on the second end plate adjacent the notch to guide the tool underneath the insert to permit the insert to be unlocked from the second end plate.

16. The implant of claim 1 further comprising an opening in the insert for receiving a tool to enable the insert to be manually disconnected from the second end plate.

17. The implant of claim 1 further comprising a tab projecting from the front end of the insert and defining a flat surface for engagement by a tool to enable the insert to be manually connected to the second end plate.

18. The implant of claim 1 further comprising a lug projecting from the front end of at least one of the end plates and adapted to be engaged by a tool to manipulate the implant relative to the vertebrae.

19. A method of implanting an intervertebral prosthesis, the method comprising:
engaging a first vertebrae with a first end plate;
engaging a second vertebrae with a second end plate;
engaging the first end plate with an insert in a manner to permit relative movement therebetween; and
engaging a projection in the insert with a groove in the second end plate to lock the insert to the second end.

20. The method of claim 19 further comprising positioning the insert at an angle to the second end plate with the projection extending in the groove and then pressing the insert towards the second end plate, to lockingly engage the insert to the second end plate.

21. The method of claim 20 wherein the projection is on the rear portion of the insert and the groove extends in the rear portion of the second end plate, and further comprising positioning the insert at an angle to the second end plate with the projection extending in the groove, and then pressing the front portion of the insert towards the second end plate.

22. The method of claim 21 wherein a projection on the sides of the insert extends in a groove on the sides of the second end plate after the step of pressing.

23. The method of claim 19 wherein the relative movement is a rotation.

24. The method of claim 19 further comprising forming a semi-spherical recess in one of the first end plate and the insert, and providing a dome on the other of the first end plate and the insert that extends in the recess to permit the relative movement.

25. An implant comprising:
a first end plate for engaging a first body portion;
a second end plate for engaging a second body portion; and
means for engaging the first end plate in a manner to permit relative movement therebetween, and for removably locking to the second end plate.

26. The implant of claim 25 wherein the means is in the form of an insert having a dome and wherein the first end plate has a recess for receiving the dome to permit relative rotation between the insert and the first end plate.

27. The implant of claim 26 wherein the insert has a projection adapted to extend a groove in the second end plate.

28. The implant of claim 27 wherein the projection lockingly engages the groove and is adapted to be manually removed from the groove to permit the insert to be disengaged from the second end plate.

29. The implant of claim 25 wherein the means is in the form of an insert having a projection adapted to extend a groove in the second end plate.

30. The implant of claim 29 wherein the projection lockingly engages the groove and is adapted to be manually removed from the groove to permit the insert to be disengaged from the second end plate.

31. A method of implanting a prosthesis, the method comprising:
attaching a first end plate to a first body portion;
attaching a second end plate to a second body portion;
engaging the first end plate with an insert in a manner to permit relative movement therebetween; and
removably locking the insert to the second end plate.
